(19) United States
(12) Patent Application Publication
(10) Pub. No.: US 2021/0369675 A1
(43) Pub. Date: Dec. 2, 2021

(54) ANTIMICROBIAL DRUG METHODS OF USE & THERAPEUTIC COMPOSITIONS

(71) Applicant: John Malcolm Hall Gregg, (US)

(72) Inventor: John Malcolm Hall Gregg, (US)

(21) Appl. No.: 16/326,433

(22) PCT Filed: Aug. 19, 2017
(86) PCT No.: PCT/US2017/047701

§ 371 Date: Feb. 19, 2019

(51) Int. Cl. A61K 31/4164; A61K 31/575; A61K 45/06

(52) U.S. Cl. CPC A61K 31/4164; A61K 45/06; A61K 31/575

(57) ABSTRACT

This invention relates to the medical use of an antimicrobial agent, racemic Ornidazole, its (R) and (S) enantiomers, or pharmaceutically acceptable salts or esters thereof, and to methods of treatment which involve treating a subject with Ornidazole. The racemic (rac)-ornidazole, its enantiomers, or pharmaceutically acceptable salts or esters thereof, may be used in combination with other actives, and in particular, beta lactam antibiotics combined with beta lactamase inhibitors, like co-amoxiclay. The invention also relates to pharmaceutical formulations and compositions comprising (rac)-ornidazole, (R)-ornidazole, (S)-ornidazole, or pharmaceutically acceptable salts or esters thereof, and/or other actives and their use with bio-threat pathogens that utilize a mechanism of tolerance or resistance by facultatively switching from aerobic to anaerobic confirmations and by switching back and forth from planktonic to biofilm forms.

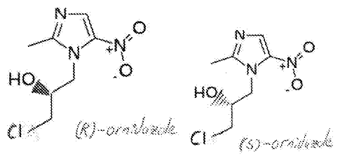

### CROSS-REFERENCE TO RELATED APPLICATIONS

This application is a national phase entry under 35 U.S.C. § 371 of International Application PCT/US2017/047701, filed Aug. 19, 2017, which claims the benefit under 35 U.S.C. § 119(e) of U.S. Provisional Application No. 62/494,762 filed Aug. 20, 2016, all of which are herein incorporated by reference in their entireties. This application further relates to U.S. Provisional Application Ser. No. 62/124,467 filed Dec. 20, 2014; U.S. Provisional Application Ser. No. 62/124,468, filed Dec. 20, 2014, U.S. Provisional Application Ser. No. 62/124,469, filed Dec. 20, 2014, U.S. Provisional Application Ser. No. 62/124,470, filed Dec. 20, 2014, U.S. Provisional Application Ser. No. 62/124,471, filed Dec. 20, 2014,U.S. Provisional Application Ser. No. 62/124,472, filed Dec. 20, 2014, U.S. patent application Ser. No. 14/967,542, filed Dec. 14, 2015, all of which are herein incorporated by reference in their entireties.

### BACKGROUND

ORNIDAZOLE is a nitroimidazole anti-infective. Its systematic (IUPAC) name is 1-chloro-3-(2-methyl-5-nitro-1H-imidazol-1-yl)propan-2-ol and its chemical formula is C₇H₁₀CIN₃O₃. Ornidazole contains a single chiral centre and thus exists as either the S-enantiomer or the R-enantiomer.

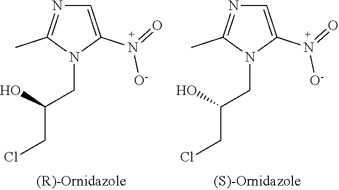

Both the (R)-ornidazole enantiomer (hereinafter referred to as, “(R)-ornidazole”), and the (S)-ornidazole enantiomer (hereinafter referred to as, “(S)-ornidazole”) have both different and unique individual spectrums of activity as well as pharmacological & safety advantages over racemic ornidazole (rac)-ornidazole). (R)-ornidazole, (S)-ornidazole, and (rac)-ornidazole are new chemical entities (NCEs) in the United States and have not been marketed in this jurisdiction or cleared by the Food & Drug Administration (FDA).

The beneficial properties of (R)-ornidazole, (S)-ornidazole, and (rac)-ornidazole include a favorable pharmacokinetic and pharmacodynamic profile and high degree of susceptibility to pathogenic strains of bacteria when the right (minimum inhibitory or bactericidal) drug concentrations are employed at the site of infections. In addition, (R)-ornidazole, (S)-ornidazole, and (rac)-ornidazole are effective at treating and eradicating bacterial biofilms of many pathogens that have sensitivity in their planktonic forms at lower MICs than the biofilm form. Many bacterial species are inducible facultative morphologs. An inducible facultative morpholog species can reversibly change configuration from planktonic to biofilm forms and morphological intermediates (such as round body/cystic/ and spore forms) based on environmental conditions and through quorum sensing mediated by chemical signalling.

These inducible morphologies, e.g., biofilms and their intermediate forms, have MICs that can be orders of magnitude higher than the planktonic forms of the bacteria. The lethal dose of the antibiotics for bacteria in the biofilm form is called the “minimal bacterial eradication concentration” or “MBEC”. The ability of the bacteria to shift into a biofilm form serves as an antimicrobial resistance (AMR) mechanism for usually adminstered dosages of antimicrobials.

(R)-ornidazole, (S)-ornidazole, and (rac)-ornidazole are highly effective in treating biofilms bacterial configurations that these bacteria utilize to combat therapeutics and maintain recurrence reservoirs. The microorganisms in biofilms live in a self-produced matrix of hydrated extracellular polymeric substances (EPS) that form their immediate environment. EPS are mainly polysaccharides, proteins, nucleic acids and lipids; they provide the mechanical stability of biofilms, mediate their adhesion to surfaces and form a cohesive, three-dimensional polymer network that interconnects and transiently immobilizes biofilm cells. In addition, the biofilm matrix acts as an external digestive system by keeping extracellular enzymes close to the cells, enabling them to metabolize dissolved, colloidal and solid biopolymers.

Planktonic isolates are susceptible to common antibiotics. Strains in biofilms and anaerobic configurations are markedly resistant to many antimicrobial agents. However, many of these biofilms and persister cells within the biofilm are sensitive to (R)-ornidazole, (S)-ornidazole, and (rac)-ornidazole when exposed to a MBEC and when used in combination with monosaccharides and oligosaccharides that increase the metabolic activity of the cells within the biofilm increasing antimicrobial uptake and toxicity.

Thus, the use of (R)-ornidazole, (S)-ornidazole, and (rac)-ornidazole may provide a number of advantages including, for example, formulations targeting the site of infections or dysbiosis/bacterial overgrowth in biofilms or formulations utilizing smaller amounts of the active drug than alternative treatments, so that patients can ingest smaller tablets or capsules, or allowing the (R)-ornidazole, (S)-ornidazole, and (rac)-ornidazole to be combined with other active agent(s) in a single unit dosage form; allowing more convenient dosing schedules and increasing patient compliance. For example, (R)-ornidazole, (S)-ornidazole, and (rac)-ornidazole are rapidly absorbed after oral administration and have a longer terminal elimination half-life (approximately 14-18 hours) than commonly used drugs in the imidazole class. Accordingly these drugs may be administered less frequently and/or at a lower dose, thus improving patient compliance while providing a therapeutically effective treatment of an infection.

Additionally, (R)-ornidazole, (S)-ornidazole, and (rac)-ornidazole are more active than metronidazole, another nitro-imidazole used therapeutically, for many anaerobic and aerobic bacterial strains (including both Gram positive and Gram negative strains). Even against strains in which (R)-ornidazole, (S)-ornidazole, and (rac)-ornidazole are as active, or even less active than metronidazole, they still offer other benefits.

(R)-ornidazole, (S)-ornidazole, and (rac)-ornidazole are active against both susceptible and resistant strains of anaerobic or aerobic Gram negative bacteria, or Gram positive bacteria. Resistant strains of a specific bacteria are strains of that bacteria which are resistant to one or more drugs normally used to treat bacterial infections, e.g. strains of bacteria which are resistant to metronidazole.

Many biological warfare agent (BWA) pathogens use anaerobic respiration to evade antibiotics. The most difficult to treat BWA pathogens, including B. pseudomallei and B. melitensis, adapt to changes in the environment to exploit available niches. These bacteria are inducible facultative respiralogs that can reversibly change metabolism from aerobic to anaerobic respiration as an AMR mechanism. B. pseudomallei and B. melitensis normally grow aerobically; however, in infected humans and animals they survive anaerobically within hypoxic lesions and can re-emerge after long periods of antibiotic treatment targeting the aerobic state. In culture, it has been demonstrated that these bacteria can live anaerobically in a nitrate containing complex medium.

Conventional antibiotics, such as ceftazidime, trimethoprim-sulfamethoxazole, and chloramphenicol, are not effective under anaerobic conditions in vitro with B. pseudomallei and B. melitensis. However, the anaerobic B. pseudomallei and B. melitensis respiralog forms are exquisitely sensitive to some nitro-imidazoles, making racemic ornidazole and its enanatiomers, ideal potential candidates for combination therapy preventing disease recurrence by eliminating bacteria from anaerobic lesion reservoirs. Indeed, the inventor has tested racemic ornidazole under anaerobic conditions and has shown inhibition of B. pseudomallei and B. melitensis at therapeutic concentrations ornidazole.

Both anaerobic bacteria and protozoal pathogens activate nitro-imidazole compounds utilizing the nitrogen reductases that are necessary for anaerobic respiration. Activation results in the formation of highly reactive nitrogen/oxygen free radical compounds that kill the pathogen. Facultative anaerobic bacteria utilize a different respiration pathway where nitrate is used as the terminal electron acceptor in place of oxygen. In aerobic situations, nitro-imidazoles are not effective against these bacteria.

The inventor's studies show that when B. pseudomallei and B. melitensis are grown under anaerobic conditions, they are effectively inhibited by nitro-imidazoles, including ornidazole. This is likely due to increased expression of bacterial nitrogen reductases, which metabolize the nitro-imidazoles into locally highly toxic free radicals.

Many BWA pathogens use anaerobic respiration to evade antibiotics. The most difficult to treat BWA pathogens, including B. pseudomallei and B. melitensis, adapt to changes in the environment to exploit available niches. These bacteria are inducible facultative respiralogs that can reversibly change configuration from aerobic to anaerobic respiration producing an AMR mechanism.

B. pseudomallei and B. melitensis normally grow aerobically but in human and animal infections survive anaerobically within hypoxic lesions and can re-emerge with aerobic respiration even after long periods of antibiotic treatment targeting the aerobic state. In culture, these bacteria can live anaerobically in nitrate containing complex medium.

Both anaerobic bacteria and protozoal pathogens activate nitro-imidazole compounds utilizing the nitrogen reductases that are necessary for anaerobic respiration. The result is the formation of highly reactive nitrogen/oxygen free radical compounds, which kill the bacteria. In the anaerobic state, facultative anaerobic bacteria utilize a respiration pathway where nitrate is used as the terminal electron acceptor in place of oxygen. Thus, in the aerobic state, nitro-imidazoles are not effective against these bacteria.

As previously stated, the inventor's studies and other published studies show that when B. pseudomallei is grown under anaerobic conditions, it is effectively inhibited by some nitro-imidazoles, and the present invention shows that this includes ornidazole. This is likely due to increased expression of bacterial nitrogen reductases, which metabolize the nitro-imidazoles into locally highly toxic free radicals.

During infection in human & mammalian models, bacteria encounter environments with varying oxygen levels. Abscesses, which are particularly common in B. pseudomallei infections, are known to be anaerobic environments and can thus serve as sanctuary bacterial reservoirs.

Ornidazole is effective in vitro against the anaerobic configurations of B. pseudomallei and B. melitensis and thus could be used in combination with other antibiotics that are effective against these bacteria in aerobic respiration configuration. This AMR pathway is common in a number of difficult to treat biothreat pathogens including those causing anthrax, Glanders, brucellosis and Melioidosis.

Current formulations of ornidazole are not gastro-retentive. To optimize therapy for both emerging threat pathogens like C. difficile, as well as BWA pathogens, the current inventions include immediate release, slightly delayed release, and gastro-retentive formulations that could be given alone or combined for different infection mixes.

BWA pathogens that are weaponized are often deployed in aerosolized forms for battlefield use. When volatile weaponized BWA aerosols are deployed, it is likely, based on studies done in animal models, that the bacteria will both be ingested, ending up in high concentrations in the GI system, as well as being inhaled, setting up lung and respiratory infections.

Based on the pharmaceutics of 5-nitroimidazole antibiotics, unique characteristics of ornidazole, new formulation technology, and improved administration, the present invention with a new gastro-tentative formulation demonstrates highly significant improvements in efficacy (cure rates and recurrence rates), compared to Vancocin (oral vancomycin), Flagyl (metronidazole) and Dificid (fidaxomicin). The new gastro-retentive formulation is a GI system restricted sustained release formulation that delivers the bulk of the drug at the colon to create a clinically relevant MIC at the luminal, mucosal, and epithelial layers at the site of infection.

The microgranule delayed release and gastro-retentive formulation for ornidazole encompassed in the present invention provides adequate drug concentrations in both the GI tract and systemic circulation. Ornidazole, like other nitro-imidazoles, is a Biopharmaceutics Classification System (BCS) Class 1 compound. This means that the drug is almost entirely, and almost immediately, absorbed from the stomach into systemic circulation. This is a big advantage in the BWA setting for aerosolized inhaled pathogens because of the rapid rate of high levels of drug in systemic circulation that can then penetrate different tissue compartment where the bacteria are present. Ornidazole also has a particularly long half-life to maintain these levels for an extended period of time with a larger area under the curve (AUC).

Systemic circulation alone, is a disadvantage, in cases where the pathogen is in the colon or small intestines because the drug must go through systemic circulation and then pass from the bloodstream through the GI organ epithelium, its mucosa, and into the lumen at a sufficient concentration to inhibit or kill the bacteria. In the case of C. difficile and a frequently used nitro-imidazole, metronidazole, this concentration is barely high enough to have some effect on planktonic C. difficile in the intestinal and colonic lumen.

In the present invention, the gastro-retentive formulation of ornidazole, in combination with the systemic microgranule formulation, has the API moving from the lumen towards the epithelium and also in the other direction, with the systemic delayed release formulation to maximize concentrations for all layers and compartments. In the present invention the systemic delayed release dual formulation will protect warfighters from ingested BWA's by delivering drug to relevant compartments at efficacious concentrations and working effectively on bacteria that are in both planktonic and biofilm and spore/round/hard body forms as well as different aerobic and anaerobic confirmations.

While initial “cure” rates with Ornidazole in the present invention, (producing clinically relevant reductions of planktonic bacteria in overgrowth infections), are important (because this reduces the likelihood of recurrence), what is also important is the strategy of using a co-administered agents to make persister cells and quiescent cells in biofilms susceptible to ornidazole to make the cure rates even higher and more durable, and the co-administration of primary bile acids, including Ursodiol (ursodeoxycholic acid), also in a gastro-retentive formulation to reach the colon, to prevent spores from bacteria like C. difficile from causing recurrences upon exposure.

The present invention included the combination use of agents to make bacteria cells in biofilms more susceptible to Ornidazole as well as gastro-retentive formulations of a secondary bile acid, including Ursodiol, to make the colonic microenvironment inhospitable to the germination of spores from bacterial species like C. difficile.

Many types of bacteria, including but not limited to C. difficile, B. melitensis, B mallei, and B. Pseudomallei, exist in a number of morphological forms, including planktonic form, round/hard body form, spore form, and biofilm form. Cells in planktonic form tend to be the most sensitive to antibiotics and respond to the lowest minimum inhibitory concentrations of the different morphologies. Bacteria in round/hard body and spore forms are much less sensitive to antibiotics and require significantly higher concentrations to inhibit replication. Bacteria in biofilm configurations are also less sensitive to antibiotics. Some of the bacterial cells in the biofilms are protected by the EPS matrix, others, often including persister cells, are metabolically inactive or relatively metabolically inactive and do not take up introduced antimicrobial agents.

Cells in biofilms have sensors which direct their movement back to planktonic configurations and also begin the process of making cells in biofilms more metabolically active in the presence of sugars and other growth substrate compounds that are co-administered with ornidazole, in one embodiment of the present invention. In addition, some of these co-administered compounds make the pH more basic (creating a better environment for bacterial growth), in GI microbiomes.

In the colonic and small intestinal microbiomes, much of the flora, which are commensal constitutive residents that exist symbiotically with host holobiont, have a main food/nutrient substrate as primary bile acids, including cholic acid and chenodeoxycholic acid. Commensal bacteria maintain their compositional equilibrium of taxonomic and relative abundance by processing primary bile acids into secondary bile acids. The primary bile acids are a major prebiotic substrate for these bacteria while the secondary bile acids have use for the host in digestion, but are not conducive to growth of many types of bacteria or the germination of their spores.

In the case of C. difficile, it has been demonstrated that toxigenic strains are kept in check that are normally crowded out through competitive inhibition from other bacterial species when there is an abundance of primary bile acids that support the growth of competitive bacteria.

When stable systems of commensal bacteria in the human small intestines and colon are disrupted by wide-spectrum antibiotics, greatly diminishing their compositional diversity and relative abundance, there is a lack of bacterial competition and a surplus of primary bile acids that arises that enables an overgrowth of C. difficile. If the overgrowth of C. difficile is with toxigenic strains, the results can be serious diarrhea related morbidity and mortality in the human population.

Merely reducing the population of planktonic toxigenic C. difficile through the use of antibiotics, including Ornidazole, is often not enough because there is a rich growth substrate remaining of primary bile acids, including cholic acid and chenodeoxycholic acid. In addition, an abundance of primary bile acids enables the germination of spores of C. difficile that patients who have had initial infections are often exposed to, causing recurrences of diarrhea producing infections.

The microbiota that were largely eliminated or greatly reduced in abundance by the initial use of a wide spectrum antibiotic (particularly those antibiotics with activity against aerobes and facultative anaerobes), played an ecological niche role of processing primary bile acids to more than 20 types of secondary bile acids. These secondary bile acids, when present in significant quantities, are not conducive to spore germination by bacteria like C. difficile.

It has been demonstrated in experiments with enemas delivering secondary bile acids that changing the balance of bile acids from mainly primary bile acids to mainly secondary bile acids, inhibits the germination of spore forming bacteria, like C. difficile, that need environments with mainly primary bile acids. The equilibrium of bile acids can be restored by means including fecal microbiota transplant [FMT] (whereby the ecology of primary bile acid processing by a wide range of bacteria is replaced), or by adding an abundance of exogenous secondary bile acids to the system until a stable microbiome can be established through natural community re-assembly based on autologous microbiome resilience.

The secondary bile acid, Ursodiol (brand name “Actigall”) is a now generic drug that is indicated in the United States for the treatment of gallstones. The label for this product states that 90% of the drug in its current 300 mg capsule formulation is absorbed in the small intestines (“small bowel”) and less than 10% of the remaining drug reaches the colon. In the present invention, Ursodiol is reformulated to have the bulk of the drug reach the colon and only a small percentage by released in the small bowel.

Thus, the present invention encompasses the co-administration of Ornidazole with secondary bile acids in a gastro-retentive formulation, including but not limited to Ursodiol or a combination of the 20 secondary bile acids that have been identified, in a proprietary orally delivered gastro-tentative formulation that will reach the distal parts of the small intestines and the entire colon.

### SUMMARY OF THE INVENTION

The present invention relates to methods of treating and/or reducing the incidence of various diseases by administering to a subject in need thereof a therapeutically effective amount of (R)-ornidazole, (S)-ornidazole, and (rac)-ornidazole, or pharmaceutically acceptable salts or esters thereof. In some embodiments, (R)-ornidazole and (S)-ornidazole, or pharmaceutically acceptable salts or esters thereof, have an enantiomeric purity of at least about 50% enantiomeric excess (ee), at least about 60% enantiomeric excess (ee), at least about 70% enantiomeric excess (ee), at least about 80% enantiomeric excess (ee), at least about 90% enantiomeric excess (ee), at least about 95% enantiomeric excess (ee), at least about 96% enantiomeric excess (ee), at least about 97% enantiomeric excess (ee), at least about 98% enantiomeric excess (ee), or at least about 99% enantiomeric excess (ee).

The present invention includes the co-administration of Ornidazole with biofilm busters and biofilm metabolic activators for quiescent and metabolically inactive cells including but not limited to persisters.

The present invention includes the co-administration with antibiotics, including but not limited to Ornidazole and its enantiomers, with gastro-retentive formulations of secondary bile acid products including, but not limited to, Ursodiol, lithocholic acid, and deoxycholic acid.

In an embodiment, the secondary bile acid products including, but not limited to, Ursodiol, lithocholic acid, and deoxycholic acid are co-administered with an Ornidazole product, selected from (R)-ornidazole, (S)-ornidazole, and (rac)-ornidazole, for use in treating toxigenic Clostridium difficile infections.

The methods of the present invention also include treating a disease associated with a respiratory, gastrointestinal, or systemic infection; the method comprising administering to a subject in need thereof a therapeutically effective amount of (R)-ornidazole, (S)-ornidazole, and (rac)-ornidazole, or pharmaceutically acceptable salts or esters thereof. In many cases, the therapeutically effective amount of (R)-ornidazole, (S)-ornidazole, and (rac)-ornidazole, or pharmaceutically acceptable salts or esters thereof, for treating the planktonic form of relevant bacteria is lower than the amount necessary to achieve a higher concentration that is the MBEC for a biofilm of that bacteria. Another words, the microbiological breakpoints are different and a totally different dosing strategy is needed based on the assessment of the MBEC rather than the in-vitro planktonic MIC. In this context, when discussing the therapeutically effective amount of (R)-ornidazole, (S)-ornidazole, and (rac)-ornidazole, it is meant the amount necessary to treat and kill the bacteria within the biofilm, not merely the planktonic bacteria, and this may sometimes require the co-administration of an oligosaccharide or monosaccharide to increase the metabolism of the bacteria within the biofilm to make (R)-ornidazole, (S)-ornidazole, and (rac)-ornidazole more effective.

In a specific embodiment, the diseases treated by (R)-ornidazole, (S)-ornidazole, and (rac)-ornidazole are caused by a bacterial infection.

Another embodiment of the invention includes a method of treating a disease associated with a respiratory, gastrointestinal, or systemic infection; the method comprising administering to a subject in need thereof a therapeutically effective amount of (R)-ornidazole, (S)-ornidazole, and (rac)-ornidazole, or pharmaceutically acceptable salts or esters thereof.

In another specific embodiment, the disease is one or more diseases selected from the group consisting of “biothreat” pathogens including, but not limited to Brucella melitensis, Bacillus anthracis, and Yersinia pestis.

In an embodiment, the (R)-ornidazole, (S)-ornidazole, and (rac)-ornidazole are for use in treating protozoa infections. In a further embodiment, the protozoa infection is selected from trichomoniasis.

(R)-ornidazole, (S)-ornidazole, and (rac)-ornidazole may also be used in treating avian trichomoniasis, also known as “Frounce” and “Canker”. The trichomoniasis is caused by Trichomonas gallinae.

In another embodiment, the one or more other antibiotics for the respiratory, gastrointestinal, and systemic infections are selected from tetracycline antibiotics, macrolide antibiotics, quinolone antibiotics, β-lactam antibiotics and penem antibiotics, for co-administration/combination with one of the Ornidazole forms of (R)-ornidazole, (S)-ornidazole, or (rac)-ornidazole.

(R)-ornidazole, (S)-ornidazole, and (rac)-ornidazole may also be used in treating both planktonic and biofilm abdominal and intra-abdominal infections, e.g. severe abdominal and intra-abdominal infections in combination with one or more other antibiotics for gastrointestinal infections. These antibiotics are selected from tetracycline antibiotics, macrolide antibiotics, quinolone antibiotics, β-lactam antibiotics and penem antibiotics.

Abdominal and intra-abdominal infections are mixed infections of gram positive and gram negative aerobic and anaerobic bacteria that typically need antibiotics from several classes to cover all of the potentially pathogenic organism. Specific examples of such abdominal and intra-abdominal infections caused by bacterial biofilms as well as planktonic morphologies include peritonitis, intra-abdominal abscess, and liver abscess. (R)-ornidazole is highly active against both planktonic forms and biofilms of bacterial species including Brucella melitensis, Bacillus anthracis, and Yersinia pestis. These bacterial species can be associated with the above mentioned abdominal and intra-abdominal infections when weaponized and used as bio-threat agents. In particular, (R)-ornidazole, (S)-ornidazole, and (rac)-ornidazole have excellent activity against biofilms of these pathogens in anaerobic form and bacteria like this that are facultative in their respiration mechanism and morphology.

Antimicrobials including tetracycline antibiotics, macrolide antibiotics, quinolone antibiotics, β-lactam antibiotics and penem antibiotics will be active against the Brucella melitensis, Bacillus anthracis, and Yersinia pestis bacteria while it is in an aerobic format for its respiration, while (R)-ornidazole, (S)-ornidazole, and (rac)-ornidazole will be active against the bacteria while they are in an anaerobic format for their respiration.

The methods of the present invention also include treating a disease, the method comprising administering to a subject in need thereof a therapeutically effective amount of (R)-ornidazole, (S)-ornidazole, and (rac)-ornidazole, or pharmaceutically acceptable salts or esters thereof; wherein the disease is Brucellosis, anthrax, and plague.

In another embodiment, the (R)-ornidazole, (S)-ornidazole, and (rac)-ornidazole, or pharmaceutically acceptable salts or esters thereof, are used to treat Clostridial infections caused by C. difficile and C. perfringens, bacteria that affects humans and other animals, and at least some of the bacteria are a spore-like small cell variant, and are obligate intracellular pathogens.

In another embodiment, (R)-ornidazole, (S)-ornidazole, and (rac)-ornidazole, or pharmaceutically acceptable salts or esters thereof, that is used to treat Brucella melitensis, Bacillus anthracis, and Yersinia pestis is for use in combination with one or more antibiotics, the antibiotics being selected from β-lactam antibiotics, tetracycline antibiotics, penem antibiotics, quinolone antibiotics and macrolide antibiotics that can contribute to the killing of the Brucella melitensis, Bacillus anthracis, and Yersinia pestis bacteria.

### DETAILED DESCRIPTION OF THE INVENTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

In any aspect or embodiment of the invention described in this specification, the (R)-ornidazole may be in the form of a pharmaceutically acceptable salt (e.g. the HCl salt) or ester. Alternatively, the (R)-ornidazole may be present as a free base, i.e. not in the form of a salt. The (R)-ornidazole, or pharmaceutically active salt or ester thereof, may be in the form of a hydrate.

Likewise, many of the embodiments of the invention are concerned with combinations of (R)-ornidazole, (S)-ornidazole, and (rac)-ornidazole with one or more other active agents. Where appropriate, and irrespective of whether (R)-ornidazole, (S)-ornidazole, and (rac)-ornidazole are in the form of a pharmaceutically acceptable salts, any one or more of the other active agents may be in the form of a pharmaceutically acceptable salt.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

It is intended that the aspects and embodiments of this invention encompasses (R)-ornidazole and/or any other active agent in all solid forms, including amorphous forms, as well as crystalline forms, and polymorphs thereof.

By (S) enantiomer is intended to mean that enantiomer which produces a positive optical rotation. This has been shown, through independent synthesis, to be the (S)-enantiomer and is identified as such throughout this specification. In the unlikely event that this assignment has been done in error, this specification is directed to (+)-Ornidazole.

This invention relates to various uses and syntheses of (R)-ornidazole. In this context (R)-ornidazole is not intended to refer only to pure (S)(+)-Ornidazole but also to Ornidazole in which the (S)(+)-enantiomer predominates over the (R)(−)-enantiomer. Thus, the term (S)(+)-Ornidazole also includes mixtures in which there is a small amount (e.g. less than 10% by weight, e.g. less than 5% by weight) of (R)(−)-Ornidazole.

The term “(S)(+)-Ornidazole” thus includes (S)(+)-Ornidazole by itself or when it is available in an enantiomeric excess over the (R)(−)-Ornidazole enantiomer.

Macrolide antibiotics are antibiotics which comprise a large (e.g. 14-, 15- or 16membered) macrocyclic lactone ring. Exemplary macrolide antibiotics include: dirithromycin, roxithromycin, telithromycin, erythromycin, clarithromycin, & azithromycin and in particular erythromycin, clarithromycin, & azithromycin.

β-Lactam antibiotics are antibiotics in which the structure features a ⊖-lactam moiety. They include cephalosporins (e.g.corecefazolin, cefacetrile, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefbuperazone, cefuroxime, cefuzonam, cefoxitin, cefotetan, cefmetazole, flomoxef, loracarbef, cefixime, ceftazidime, ceftriaxone, cefcapene, cefdaloxime, cefetamet, cefmenoxime, cefodizime, cefoperazone, cefotaxime, cefpimizole, cefpiramide, cefpodoxime, cefsulodin, cefteram, ceftibuten, ceftiolene, ceftizoxime, latamoxef, cefepime, cefozopran, cefpirome, cefquinome, ceftobiprole, ceftaroline, cefdinir, cefprozil, cefalexin), penems (e.g. faropenem, biapenem, doripenem, ertapenem, imipenem, meropenem, panipenem) and penicillin derivatives (e.g. amoxillin and penicillin). Exemplary β-lactam antibiotics include amoxillin, amoxiclav, co-amoxiclav, cefazolin, cefuroxime, ceftriaxone, cefipime, ceftazidine, & cefoxitin. Further exemplary β-lactam antibiotics include penicillin & cephalosporin.

Quinolone antibiotics (which include the fluoroquinolone antibiotics) are antibiotics with a quinolone (or aza-quinolone) backbone. They include enoxacin, fleroxacin, lomefloxacin, nadifloxacin, norfloxacin, rufloxacin, balofloxacin, grepafloxacin, pazufloxacin, sparfloxacin, temafloxacin, tosufloxacin, besifloxacin, clinafloxacin, garenoxacin, gemifloxacin, gatifloxacin, sitafloxacin, trovafloxacin, prulifloxacin, ciprofloxacin, levofloxacin and ofloxacin. Exemplary quinolone antibiotics include ciprofloxacin, levofloxacin, enoxacin, fleroxacin, & ofloxacin.

Where antibiotics (and particularly quinolone antibiotics) are used in combination with (R)-ornidazole, the antibiotic will typically be administered orally.

Throughout this specification, the terms aerobic and anaerobic bacteria are used to describe the bacterial species against which (R)-ornidazole, (S)-ornidazole, and (rac)-ornidazole are active against. Bacterial species may be obligate aerobic species or they may be non-obligate aerobic species. Likewise, bacterial species can be obligate anaerobic species or non-obligate species. Sometimes, an obligate aerobic species is able to survive in anaerobic conditions by the actions of accompanying anaerobic bacteria. (R)-ornidazole is useful against biofilm infections caused by any or all of the above bacteria.

Throughout this specification the term ‘in combination’ means that either (R)-ornidazole, (S)-ornidazole, or (rac)-ornidazole and the one or more other actives are both administered to the patient over the same period of treatment. They may be administered together, i.e. at the same time. In this case they may be administered in a single formulation, (e.g. as a single tablet or capsule or sachet) or in separate formulations administered simultaneously or nearly simultaneously. Alternatively, they may be administered at separate times of day. Where the (R)-ornidazole, (S)-ornidazole, and (rac)-ornidazole and other active(s) are administered separately it is to be under stood that the timing of separate dosing is selected such that the beneficial effect of the first administered agent is not lost prior to administration of the second or further agent. Whatever the precise timing of the administration, (R)-ornidazole, (S)-ornidazole, and (rac)-ornidazole and the one or more other actives may be administered via different means, e.g. the (R)-ornidazole may be administered in an oral formulation and the other active may be administered as a topical formulation or vice versa.

The combinations of the invention provide benefits which are at least additive compared to the use of either agent alone. In many embodiments, the combinations are something more than additive e.g. synergistic compared to the use of either agent alone.

References to kits in this specification where (R)-ornidazole, (S)-ornidazole, and (rac)-ornidazole, or pharmaceutically acceptable salts or esters thereof, is used in kit form with one or more different active agents optionally further comprise instructions for the administration of the (R)-ornidazole, (S)-ornidazole, and (rac)-ornidazole, or pharmaceutically acceptable salts or esters thereof, and the other active agent(s) in the kit.

The definition of the term ‘treatment’ in this specification encompasses prophylaxis and prevention (i.e. reducing or eliminating the risk of contracting the disease). As well as meaning curing a person of the disease, ‘treatment’ also includes preventing the onset of symptoms, controlling (e.g. by slowing or eliminating) progression of disease, preventing the spread of the disease to other parts of the body and/or to other persons, reducing the spread of the disease and other facets of medical practice which will be readily understood by the person skilled in the art to fall within the meaning of the term ‘treatment’.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Formulations

For the above-mentioned therapeutic uses the dosage administered will, of course, vary with the compound employed, the mode of administration, the treatment desired and the disorder indicated. For example, if the (R)-ornidazole is administered orally, then the daily dosage of the compound of the invention may be in the range from 0.01 micrograms per kilogram body weight (pg/kg) to 100 milligrams per kilogram body weight (mg/kg). In a specific embodiment, the formulations for administration to a subject contain about 2.0 g of (R)-ornidazole, about 1.5g of (R)-ornidazole, about 1g of (R)-ornidazole, about 0.5g of (R)-ornidazole, about 0.4g of (R)-ornidazole. 0.3g of (R)-ornidazole, 0.2g of (R)-ornidazole, and about 0.1g of (R)-ornidazole. This is also true of the (S) enantiomer of Ornidazole [(S)-ornidazole] and the Ornidazole racemic mixture.

For the above-mentioned therapeutic uses the dosage of Ursodiol administered will, of course, vary with the treatment desired and the disorder indicated. For example, if Ursodiol is administered orally in capsule form for gastro-retentive delivery, then the daily dosage of the compound of the invention may be in the range from 300 mg to 1200 mg per day.

The beneficial properties of (R)-ornidazole, (S)-ornidazole, and (rac)-ornidazole include a favorable pharmacokinetic and pharmacodynamic profile. (R)-Ornidazole and (S)-ornidazole are components of rac-ornidazole and have similar, but slightly improved PK profiles. Indeed, In particular embodiments, after administration of rac-Ornidazole in a formulation, the Tₘₐₓ of rac-Ornidazole ranges about 2 hours to about 4 hours inclusive of all ranges therebetween.

In another embodiment, after administration of rac-ornidazole in a formulation, the Cₘₐₓ of rac-ornidazole ranges (after single administration) from about 9 mg/L to about 31.5 mg/L, inclusive of all ranges therebetween.

In another specific embodiment, rac-ornidazole concentrations, including the (R)-ornidazole and (S)-ornidazole components, have been measured in the colonic (8.7 mg/g) and abdominal (3.6 to 4.4mg/g) walls and epiploic fat (3.4 to 4.7 mg/g) throughout colorectal surgery in those receiving a 1 g intravenous dose for surgical prophylaxis. In another study, concentrations were measured in epiploic fat (2.48 to 4.64 mg/g) throughout liver transplantation after a 500 mg intravenous dose was given together with ceftriaxone 1 g for surgical prophylaxis. Penetration rates for this study compared with plasma concentrations ranged between 50 and 70%.

In another embodiment, after a single administration of rac-ornidazole in a formulation, the present invention provides an AUC_{0-∞} for rac-ornidazole of about 185 to about 375 mg-hr/L, and about 500 to about 511 mg-hr/L, inclusive of all ranges there between. The AUC for the (R)-ornidazole and (S)-ornidazole components are similar.

In another embodiment, after a single administration of rac-ornidazole in a formulation, the elimination half-life (T_{1/2}) of rac-ornidazole is about 14 hours to about 18 hours. The (T_{1/2}) of the (R)-ornidazole and (S)-ornidazole components are similar.

(R)-ornidazole, (S)-ornidazole, and (rac)-ornidazole, or pharmaceutically acceptable salts or esters thereof, may be used on their own but will generally be administered in the form of a pharmaceutical composition in which the (R)-ornidazole, (S)-ornidazole, and (rac)-ornidazole, or pharmaceutically acceptable salts thereof, are in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of (R)-ornidazole, (S)-ornidazole, and (rac)-ornidazole, the pharmaceutical composition which is used to administer (R)-ornidazole, (S)-ornidazole, and (rac)-ornidazole will preferably comprise from 0.05 to 99 % w (per cent by weight) (R)-ornidazole, (S)-ornidazole, and (rac)-ornidazole, more preferably from 0.05 to 80%w (R)-ornidazole, (S)-ornidazole, and (rac)-ornidazole, still more preferably from 0.10 to 70%w (R)-ornidazole, (S)-ornidazole, and (rac)-ornidazole, and even more preferably from 0.10 to 50%w (R)-ornidazole, (S)-ornidazole, and (rac)-ornidazole, of active ingredient, all percentages by weight being based on total composition.

In many of the embodiments of the invention, (R)-ornidazole, (S)-ornidazole, and (rac)-ornidazole are used in combination with other active agents (e.g. antibiotics, antifungal, anti-inflammatory agent, proton pump inhibitors etc.). Depending on the mode of administration of the other active agent, the pharmaceutical composition used to administer the other active agent (which may or may not be the same pharmaceutical composition which is used to administer (R)-ornidazole, (S)-ornidazole, and (rac)-ornidazole will preferably comprise from 0.05 to 99%w (per cent by weight) of the other active agent, more preferably from 0.05 to 80%w of the other active agent, still more preferably from 0.10 to 70%w of the other active agent, and even more preferably from 0.10 to 50%w of the other active agent, of active ingredient, all percentages by weight being based on total composition.

The pharmaceutical compositions may be administered topically (e.g. to the vagina) in the form, e.g., of creams, gels, lotions, solutions, suspensions, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

For oral administration (R)-ornidazole, (S)-ornidazole, and (rac)-ornidazole and/or one or more other active agents may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, (R)-ornidazole, (S)-ornidazole, and (rac)-ornidazole and/or one or more other active agents may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration (R)-ornidazole, (S)-ornidazole, and (rac)-ornidazole and/or one or more other active agents may be administered as a sterile aqueous or oily solution. Parenteral formulations are particularly suitable for patients suffering from a severe infections. The person skilled in the art would be well aware of what differentiates a serious infection from a non-serious infection. By way of example, severe infections include those which render the patient unable to take (R)-ornidazole, (S)-ornidazole, and (rac)-ornidazole orally, e.g. infections which render the patient unconscious, emetic, weak, delirious etc. The HCl salt of (R)-ornidazole, (S)-ornidazole, and (rac)-ornidazole are particularly suitable for parenteral administration, e.g. for the treatment of severe infections.

The size of the dose for therapeutic or prophylactic purposes of (R)-ornidazole, (S)-ornidazole, and (rac)-ornidazole and/or one or more other active agents will naturally vary according to the nature and severity of the conditions, the age and sex of the animal or patient and the route of administration, according to well-known principles of medicine.

Dosage levels, dose frequency, and treatment durations of (R)-ornidazole, (S)-ornidazole, and (rac)-ornidazole are expected to differ depending on the formulation and clinical indication, age, and co-morbid medical conditions of the patient. In adult patients, as a single agent monotherapy, the daily dose of orally, parentally or rectally administered forms of (R)-ornidazole, (S)-ornidazole, and (rac)-ornidazole are expected to vary from 0.25 g/day-8.0 g/day. Downward dose adjustments from these levels are likely to be needed in infants (0-2 years of age), children (2-18 years of age), and elderly patients (greater than 65 years of age), as well as individuals with renal or liver disease, and upward dose adjustments may be necessary in obese individuals. In adult patients, as a single agent monotherapy, the concentration of topically or vaginally administered forms of (R)-ornidazole, (S)-ornidazole, and (rac)-ornidazole are expected to vary from 0.10-4.0 g/day with the concentration of (R)-ornidazole, (S)-ornidazole, and (rac)-ornidazole in the emollient varying between 0.25%-5%. As a single agent monotherapy, the standard duration of (R)-ornidazole, (S)-ornidazole, and (rac)-ornidazole treatment is expected to vary between one and seven days for most clinical indications. It may be necessary to extend the duration of treatment beyond seven days in instances of recurrent infections or infections associated with tissues or implanted materials to which there is poor blood supply including bones/joints, respiratory tract, endocardium, and dental tissues.

When (R)-ornidazole, (S)-ornidazole, and (rac)-ornidazole is combined with other medications in fixed dose combinations treatments, the daily dose of orally, parentally, topically, vaginally, or rectally administered forms of (R)-ornidazole, (S)-ornidazole, and (rac)-ornidazole are expected to vary from 0.001 g/day-4.0 g/day.

A specific example of (R)-ornidazole, (S)-ornidazole, and (rac)-ornidazole oral sachet formulations contains (R)-ornidazole, (S)-ornidazole, and (rac)-ornidazole and the following excipients: sugar spheres, Povidone, Polyethylene glycol 4000, Aerosil 200, Talc and Eudragit NE30D. The formulation weighs 4.2 g and contains 2 g of (R)-ornidazole, i.e. the formulation contains about 48% (R)-ornidazole by weight.

Treatment of Gastrointestinal Diseases

In an embodiment, (R)-ornidazole, (S)-ornidazole, and (rac)-ornidazole may also be used in treating gastrointestinal infections.

In an embodiment, the (R)-ornidazole, (S)-ornidazole, and (rac)-ornidazole are for use in treating a condition selected from gastrointestinal infections caused by Brucella melitensis, Bacillus anthracis, Yersinia pestis, and germinating spores of Clostridium difficile.

In an embodiment, the (R)-ornidazole, (S)-ornidazole, and (rac)-ornidazole co-administered with a secondary bile acid, including, but not limited to Ursodiol, are for use in treating a condition selected from initial episode and recurrent Clostridium difficile infection and associated diarrhea.

Treatment of Abdominal and Intra-Abdominal Infections

In one group of embodiments, the present invention provides the use of (R)-ornidazole, (S)-ornidazole, and (rac)-ornidazole in a combination product (e.g. a fixed dose combination product) with a quinolone (including, but not limited to ciprofloxacin, levofloxacin, enoxacin, fleroxacin, & ofloxacin) antibiotic for the prophylaxis and treatment of abdominal and intra-abdominal infections (including severe abdominal and intra-abdominal infections, e.g. peritonitis, intra-abdominal abscess, liver abscess) caused by Brucella melitensis, B. pseudomallei, B. anthracis, Y. pestis and B. mallei.

In another group of embodiments, are provided formulations (e.g. oral or parenteral formulations) comprising (R)-ornidazole, (S)-ornidazole, and (rac)-ornidazole, alone or in combination with other antibiotics in the quinolone, beta lactam, and macrolide classes, for use in treating abdominal and intra-abdominal infections (including severe abdominal and intra-abdominal infections, e.g. peritonitis, intra-abdominal abscess, liver abscess) caused by Brucella melitensis, B. pseudomallei, B. anthracis, Y. pestis and B. mallei.

In yet another group of embodiments, is provided the use of (R)-ornidazole, (S)-ornidazole, and (rac)-ornidazole in a combination product (e.g. a fixed dose combination product) with a beta lactam antibiotic for the prophylaxis and treatment of abdominal and intra-abdominal infections (including severe abdominal and intra-abdominal infections, e.g. peritonitis, intra-abdominal abscess, liver abscess) caused by Brucella melitensis, B. pseudomallei, B. anthracis, Y. pestis, and B. mallei.

Treatment of Respiratory Infections

In one group of embodiments, the present invention provides the use of (R)-ornidazole, (S)-ornidazole, and (rac)-ornidazole in a combination product (e.g. a fixed dose combination product) with a quinolone (including, but not limited to ciprofloxacin, levofloxacin, enoxacin, fleroxacin, & ofloxacin) antibiotic for the prophylaxis and treatment of respiratory infections (including upper and lower respiratory tract infections, e.g. pharyngitis, bronchitis, and pneumonia) caused by Brucella melitensis, B. pseudomallei, B. anthracis, Y. pestis and B. mallei.

In another group of embodiments, are provided formulations (e.g. oral or parenteral formulations) comprising (R)-ornidazole, (S)-ornidazole, and (rac)-ornidazole, alone or in combination with other antibiotics in the quinolone, beta lactam, and macrolide classes, for use in treating respiratory infections (including upper and lower respiratory tract infections, e.g. pharyngitis, bronchitis, and pneumonia) caused by Brucella melitensis, B. pseudomallei, B. anthracis, Y. pestis and B. mallei.

In yet another group of embodiments, is provided the use of (R)-ornidazole, (S)-ornidazole, and (rac)-ornidazole in a combination product (e.g. a fixed dose combination product) with a beta lactam antibiotic for the prophylaxis and treatment of respiratory infections (including upper and lower respiratory tract infections, e.g. pharyngitis, bronchitis, and pneumonia) caused by Brucella melitensis, B. pseudomallei, B. anthracis, Y. pestis, and B. mallei.

Treatment of Avian Disease Caused by Trichomoniasis

Avian trichomoniasis, sometimes referred to as “Frounce” or “Canker” is a highly transmissible protozoal infection. The disease is caused by the single-celled protozoan parasite Trichomonas galiinae producing mechanical stress on host cells and then ingesting cell fragments after cell death.

(R)-Ornidazole and (S)-ornidazole

The biological activity of the (R) and (S) enantiomer of Ornidazole may be also enhanced against resistant strains of some of the organisms which cause the infections described in this specification. The present invention of using this compound in these settings therefore provides a meaningful clinical benefit to patients through their eradication.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference. This includes Exhibit 1: Determining the Minimum Inhibitory Concentration of Ornidazole under Anaerobic Conditions for Burkholderia pseudomallei and Brucella melitensis.

### II. REFERENCES

1. Bolton. R., Culshaw, M., (1986) Faecal metronidazole concentrations during oral and intravenous therapy for antibiotic associated colitis due to Clostridium difficile. Gut. 27: 1169-1172.
2. Dublanchet, A., Durieux, R. (1980) In vitro susceptibility of some aerobic and anaerobic bacteria to three 5-nitro-imidazole derivatives: metronidazole, ornidazole and tinidazole Ann Microbiol (Paris) January-February; 131 (1): 45-59.
3. Gallusser A., (1983) Comparative study, using 3 methods, of the sensitivity to metronidazole and ornidazole of anaerobic or related bacteria. Ann Microbiol (Paris). September-October; 134B (2): 307-21.
4. Gauthier, Y. P., et. al., (2001) Study on the pathophysiology of experimental Burkholderia pseudomallei infection in mice. FEMS Immunol Med Microbiol, 30(1): p. 53-63
5. Gelhaus, H. C., (2016) Determining the Minimum Inhibitor Concentration of Ornidazole under Anaerobic Conditions for Burkholderia pseudomallei and Brucella melitensis. Data on File, BalinBac Therapeutics, Inc. (Full report available upon request).
6. Gelhaus, H. C., et. al., (2013) Efficacy of post exposure administration of doxycycline in a murine model of inhalational Melioidosis. Sci Rep, 3: p. 1146.
7. Gorenek, L., Dizer, U., Besirbellioglu, B., Eyigun, C., Hacibektasoglu, A., Van Thiel, D., (1999) The diagnosis and treatment of Clostridium difficile in antibiotic-associated diarrhea Hepatogastroenterol January-February; 46(25): 343-348.
8. Hamad, M. A., et al., (2011) Adaptation and antibiotic tolerance of anaerobic Burkholderia pseudomallei. Antimicrob Agents Chemother. 55(7): p. 3313-23.
9. Hoppe, I., et. al., (1999) Characterization of a murine model of Melioidosis; comparison of different strains of mice, Infect Immun 67(7): p.3593-3600.
10. Lamp, K C., Freeman, C D., Klutrnan, N E., Lacy, M K., (1999) Pharmacokinetics and pharmacodynamics of the nitroimidazole antimicrobials. Clin Pharmacokinet 36:353-73.
11. Lea key, A. K., Ulett, G. C., Hirst, R. G., (1998) BALB/c and C57 mice infected with virulent Burkholderia pseudomallei provide contrasting animal models for the acute and chronic forms of human Melioidosis. Microb Pathog; 24(5) p. 269-75.
12. Lin, B., et. al., (2002) Model of differential susceptibility to mucosal Burholderia pseudomallei infection; Infect Immun: 70(2) p. 504-11.
13. Muller, M., Mode of action of metronidazole on anaerobic bacteria and protozoa. Surgery, 1983. 93(1 Pt 2): p. 165-71.
14. Nevins, C., et. al., Lethality Comparison of Three Burholderia pseudomallei Strains in BALB/c Mice: in 54th Interscience Conference of Antimicrobial Agents and Chemotherapy: Washington D.C.
15. Olsson-Liljequist, B., Nord, C., (1981) In vitro susceptibility of anaerobic bacteria to nitroimidazoles. Scand J Infect Dis Suppl. 26:42-5
16. Wust, J., (1977) Susceptibility of anaerobic bacteria to metronidazole, ornidazole, and
tinidazole and routine susceptibility testing by standardized methods. Antimicrob Agent Chemother. April 11 (4):631637.
17. Yeager, J., et. al., (2011) Characterization of the natural history of inhalational Glanders and inhalational Melioidosis in nonhuman primate models. In ASM Biodefense & Emerging Disease Research Meeting. Washington, D.C.

1. A method for the treatment or prophylaxis of a disease in a human or other animal associated with a dysbiosis of a microbial microbiome with bacteria, protozoa, and fungi in various morphological conformations, including biofilms, the method comprising administering to the human or other animal in need thereof a compound selected from the group consisting of (R)-ornidazole, (S)-ornidazole, and a racemic mixture of (R)-ornidazole and (S)-ornidazole [rac-ornidazole], including combinations and pharmaceutically acceptable salts thereof, wherein the disease is selected from the group consisting of: Respiratory tract diseases, Gastrointestinal tract disease, reproductive tract disease, protozoal disease, Brucella melitensis infections, anthrax, and plague infections.

2. The method of claim 1, wherein the respiratory, gastrointestinal, and reproductive tract diseases are selected from the group consisting of Ovine Brucellosis disease, anthrax disease caused by Bacillus anthracis, and Pneumonic and Bubonic Plague caused by Yersinia pestis.

3. The method of claim 1, wherein the gastrointestinal tract and the respiratory tract diseases are C. difficile Associated Diarrhea (CDAD), Ovine Brucellosis disease, anthrax disease caused by Bacillus anthracis, and Pneumonic and Bubonic Plague caused by Yersinia pestis infections caused by biofilms and planktonic forms of toxigenic and non-toxigenic strains of these bacteria.

4. The method of claim 1, wherein the diseases are protozoal and affect avian animals, including Frounce/Canker caused by Trichomonas gallinae.

5. The method of claim 1, comprising the administration in combination with one or more antibiotics, the antibiotics being selected from β-Iactam antibiotics (including co-amoxiclav), tetracycline antibiotics and macrolide antibiotics.

6. The method of claim 1, comprising the administration in combination with one or more gastro-retentive formulations of secondary bile acid products.

7. The method of claim 1, comprising the administration in combination with one or more gastroretentive formulations, of one or more sugars, metabolic activators, or compounds to disperse or bust biofilms.

8. The method of claim 3, wherein the infections are caused by biofilms and wherein at least some of the bacteria are present in an anaerobic conformation.

9-11 (canceled)

12. The method of claim 6, wherein the secondary bile acid products are selected from the group consisting of Ursodiol, lithocholic acid, and deoxycholic acid.

13. The method of claim 1 wherein the disease is or is caused by a protozoal infection.

14. The method of claim 13, wherein the protozoal infection is selected from Trichomonas gaffinae.

15. A method for changing the compositional abundance of bile acids in the colon in a human or other animal, comprising administering to the human or other animal in need thereof a gastro-retentive formulation of a secondary bile acid, and wherein the gastro-retentive formulation is delivered primarily to the colon.

16. The method of claim 15, wherein the gastro-retentive formulation of a secondary bile acid is selected from .
